(19) United States
(12) Patent Application Publication
(10) Pub. No.: US 2009/0052086 A1
(43) Pub. Date: Feb. 26, 2009

(54) METHOD OF MAKING HEAD SLIDER AND RESULTANT HEAD SLIDER

(75) Inventor: Hiroki Hashimoto, Kawasaki (JP)

Correspondence Address: GREER, BURNS & CRAIN, 300 S WACKER DR, 25TH FLOOR, CHICAGO, IL, 60606, US

(73) Assignee: FUJITSU LIMITED, Kawasaki-shi (JP)

(21) Appl. No.: 12/263,249

(22) Filed: Oct. 31, 2008

Related U.S. Application Data
(63) Continuation of application No. PCT/JP2006/309473, filed on May 11, 2006.

(51) Int. Cl. G11B 5/60; A61N 5/067

(52) U.S. Cl. 360/234.3; 250/492.3

(57) ABSTRACT

A laser beam is radiated to the corner of the back surface of the head slider having the front surface defining a medium-opposed surface. This method employs a laser beam radiated to the corner of the back surface of the head slider. The material thus gets molten at least partly at the corner of the head slider. The molten material then gets cured or hardened. The corner of the head slider warps back. The corner of the medium-opposed surface is in this manner chamfered. The shape of the chamfer can clearly be observed. Such chamfering process can be repeated until a desired shape is obtained. In this manner, the head slider can readily be chamfered with a high accuracy.

This application is a Continuation of International Application Serial No. PCT/JP2006/309473, filed May 11, 2006.

### BACKGROUND OF THE INVENTION

1. Field of the Invention

The present invention relates to a method of making a head slider incorporated in a hard disk drive, HDD, for example.

2. Description of the Prior Art

A head slider has a medium-opposed surface facing the surface of a hard disk, HD, at a distance, as disclosed in Japanese Patent Application Publication No. 2000-306226, for example. The corner of the medium-opposed surface of the head slider is chamfered. An inclined surface is thus formed. Even when the head slider collides against the surface of the hard disk, the inclined surface serves to reduce damages to the hard disk.

The corner of the head slider is rubbed with a faceplate, for example. A slurry containing abrasive particles is dropped on the faceplate. The slurry covers over the inclined surface. The shape of the inclined surface cannot thus be observed. The head slider has to be washed for the observation so as to obtain a desired shape. Chamfering process is troublesome.

### SUMMARY OF THE INVENTION

It is accordingly an object of the present invention to provide a method of making a head slider contributing to establishment of a chamfer, with a high accuracy, at a corner of a medium-opposed surface. It is also an object of the present invention to provide a head slider having a chamfer made according to such a method.

According to a first aspect of the present invention, there is provided a method of making a head slider, comprising at least radiating a laser beam to the corner of the back surface of the head slider having the front surface defining a medium-opposed surface.

The method employs a laser beam radiated to the corner of the back surface of the head slider. The material thus gets molten at least partly at the corner of the head slider. The molten material then gets cured or hardened. The corner of the head slider warps back. The corner of the medium-opposed surface is in this manner chamfered. The shape of the chamfer can clearly be observed. Such chamfering process can be repeated until a desired shape is obtained. In this manner, the head slider can readily be chamfered with a high accuracy.

The radiation conditions, such as duration of irradiation, energy of irradiation, the number of irradiation, and the like, of the laser beam can be specified for establishment of a desired shape. It is thus possible to chamfer the head slider with a high accuracy in accordance with the radiation conditions. Such radiation conditions enable establishment of the chamfer of a desired uniform shape on a plurality of head sliders.

No dust is generated in the process of melting the head slider. The head slider is prevented from suffering from adherence of dust to the medium-opposed surface of the head slider. On the other hand, lapping process requires abrasive grains or particles to rub off a head slider, for example. Dust is thus generated. Adhesion of the dust to the medium-opposed surface is inevitable. The medium-opposed surface gets contaminated.

The method may employ the head slider made of a material melting in response to exposure to the laser beam. The material generates shrinkage stress when the material gets cured. The laser beam may form a circular spot on the back surface of the head slider, or a line of exposure on the back surface of the head slider.

According to a second aspect of the present invention, there is provided a method of making a head slider, comprising forming a slit on the corner of the medium-opposed surface of the head slider.

When the slit is formed at the corner of the medium-opposed surface, the slit enables release of residual stress on the surface of the head slider. The corner of the medium-opposed surface is thus chamfered. The shape of the chamfer can clearly be observed. Such chamfering process can be repeated until a desired shape is obtained. In this manner, the head slider can readily be chamfered with a high accuracy.

Conditions, such as the length of the slit, the width of the slit, the position of the slit, and the like, can be specified for establishment of a desired shape. It is thus possible to chamfer the head slider with a high accuracy in accordance with the conditions. Such conditions enable establishment of the chamfer of a desired uniform shape on a plurality of slider bodies. The method may employ a focused ion beam radiated to the medium-opposed surface for establishment of the slit.

The aforementioned method serves to provide a head slider comprising: a slider body having a medium-opposed surface; and a chamfer formed at the corner of the medium-opposed surface near the outflow end of the slider body based on radiation of a laser beam. The head slider may have the chamfer defining a curved surface. The head slider may be incorporated in a storage apparatus such as a hard disk drive, for example.

### BRIEF DESCRIPTION OF THE DRAWINGS

The above and other objects, features and advantages of the present invention will become apparent from the following description of the preferred embodiments in conjunction with the accompanying drawings, wherein:

FIG. 1 is a plan view schematically illustrating the structure of a hard disk drive as a specific example of a storage apparatus;

FIG. 2 is a perspective view schematically illustrating a flying head slider according to an embodiment of the present invention;

FIG. 3 is an enlarged partial perspective view schematically illustrating radiation of a laser beam forming a circular spot on a slider body at the corner;

FIG. 4 is an enlarged partial perspective view schematically illustrating the corner of the slider body chamfered based on the molten state of the material;

FIG. 5 is an enlarged partial perspective view schematically illustrating radiation of a laser beam forming a straight line of exposure on a slider body at the corner;

FIG. 6 is an enlarged partial perspective view schematically illustrating a slit formed at the corner of the slider body;

FIG. 7 is an enlarged partial perspective view schematically illustrating the corner of the slider body chamfered based on the slits; and

FIG. 8 is a perspective view schematically illustrating a flying head slider according to another embodiment of the present invention.

### DESCRIPTION OF THE PREFERRED EMBODIMENTS

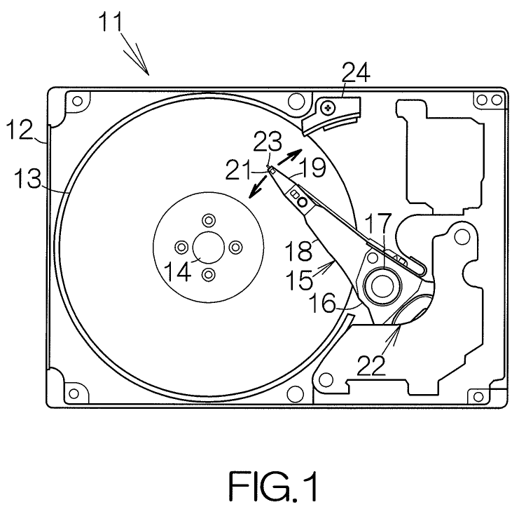

FIG. 1 schematically illustrates the structure of a hard disk drive, HDD, 11 as an example of a storage medium drive or storage apparatus. The hard disk drive 11 includes a box-shaped enclosure body 12 defining an inner space of a flat parallelepiped, for example. The enclosure body 12 may be made of a metallic material such as aluminum, for example. Molding process may be employed to form the enclosure body 12. An enclosure cover, not shown, is coupled to the enclosure body 12. The enclosure cover serves to close the opening of the inner space within the enclosure body 12. Pressing process may be employed to form the enclosure cover out of a plate material, for example.

At least one magnetic recording disk 13 as a storage medium is enclosed in the enclosure body 12. The magnetic recording disk or disks 13 are mounted on the driving shaft of a spindle motor 14. The spindle motor 14 drives the magnetic recording disk or disks 13 at a higher revolution speed such as 5,400 rpm, 7,200 rpm, 10,000 rpm, or the like.

A carriage 15 is also enclosed in the enclosure body 12. The carriage 15 includes a carriage block 16. The carriage block 16 is supported on a vertical support shaft 17 for relative rotation. Carriage arms 18 are defined in the carriage block 16. The carriage arms 18 extend in parallel in the horizontal direction from the vertical support shaft 17. The carriage block 16 may be made of aluminum, for example. Molding process may be employed to form the carriage block 16, for example.

A head suspension 19 is fixed to the tip end of the individual carriage arm 18. The head suspension 19 extends forward from the tip end of the carriage arm 18. A so-called gimbal spring, not shown, is connected to the tip end of the head suspension 19. A flying head slider 21 is fixed to the surface of the gimbal spring. The gimbal spring allows the flying head slider 21 to change its attitude relative to the head suspension 19.

A head element or electromagnetic transducer, not shown, is mounted on the individual flying head slider 21. The electromagnetic transducer includes a write element and a read element. The write element may include a thin film magnetic head designed to write magnetic bit data into the magnetic recording disk 13 by utilizing a magnetic field induced at a thin film coil pattern. The read element may include a giant magnetoresistive (GMR) element or a tunnel-junction magnetoresistive (TMR) element designed to discriminate magnetic bit data on the magnetic recording disk 13 by utilizing variation in the electric resistance of a spin valve film or a tunnel-junction film.

When the magnetic recording disk 13 rotates, the flying head slider 21 is allowed to receive an airflow generated along the rotating magnetic recording disk 13. The airflow serves to generate a positive pressure or a lift as well as a negative pressure on the flying head slider 21. The lift is balanced with the negative pressure and the urging force of the head suspension 19, so that the flying head slider 21 is allowed to keep flying above the surface of the magnetic recording disk 13 during the rotation of the magnetic recording disk 13 at a higher stability.

A power source or voice coil motor, VCM, 22 is coupled to the carriage block 16. The voice coil motor 22 serves to drive the carriage block 16 around the vertical support shaft 17. The rotation of the carriage block 16 allows the carriage arms 18 and the head suspensions 19 to swing. When the carriage arm 18 swings around the vertical support shaft 17, the flying head slider 21 is allowed to move along the radial direction of the magnetic recording disk 13. The electromagnetic transducer on the flying head slider 21 can thus be positioned right above a target recording track on the magnetic recording disk 13.

A load tab 23 is attached to the front or tip end of the head suspension 19. The load tab 23 extends forward from the head suspension 19. The load tab 23 is allowed to move in the radial direction of the magnetic recording disk 13 based on the swinging movement of the carriage arm 18. A ramp member 24 is located outside the magnetic recording disk 13 on the movement path of the load tab 23. The ramp member 24 and the load tabs 23 in combination establish a so-called load/unload mechanism. The ramp member 24 may be made of a hard plastic material, for example.

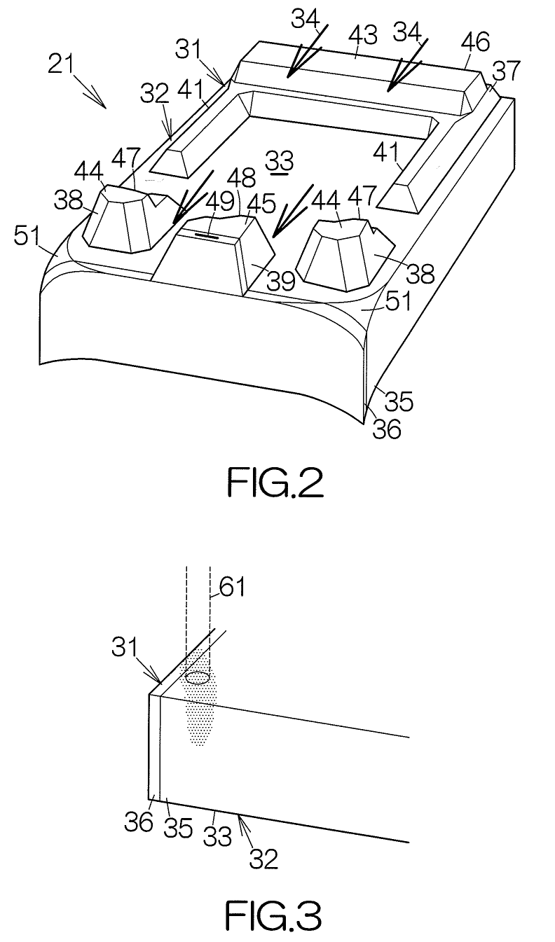

Next, a detailed description will be made on the structure of the flying head slider 21. The flying head slider 21 includes a slider body 31 in the form of a flat parallelepiped, as shown in FIG. 2, for example. A medium-opposed surface, namely a bottom surface 32, is defined over the slider body 31 so as to face the magnetic recording disk 13 at a distance. A flat base surface 33 is defined on the bottom surface 32. When the magnetic recording disk 13 rotates, airflow 34 flows along the bottom surface 32 from the inflow or front end toward the outflow or rear end of the slider body 31. The slider body 31 may include a base mass 35 made of Al₂O₃—TiC and an Al₂O₃ (alumina) film 36 overlaid on the outflow or trailing end surface of the base mass 35, for example.

A front rail 37 is formed on the bottom surface 32 of the slider body 31. The front rail 37 stands upright from the base surface 33 at a position near the upstream or inflow end of the slider body 31. The front rail 37 extends along the inflow end of the base surface 33 in the lateral direction perpendicular to the direction of the airflow 34. A pair of rear side rails 38, 38 also stand upright from the base surface 33 at positions near the downstream or outflow end of the slider body 31. The rear side rails 38 are located near the side edges of the base surface 33, respectively. A rear center rail 39 stands upright from the base surface 33 at a position between the rear side rails 38. The rear center rail 39 extends upstream in the longitudinal direction from the outflow end toward the inflow end of the base surface 33.

A pair of side rails 41, 41 are connected to the front rail 37. The side rails 41 stand upright from the base surface 33. The side rails 41, 41 extend downstream along the side edges of the base surface 33 in the longitudinal direction from the front rail 37 toward the rear side rails 38, 38, respectively. A gap is defined between the side rails 41, 41 and the corresponding rear side rails 38, 38, respectively. The gaps allow airflow to run through between the side rails 41 and the corresponding rear side rails 38, respectively. The side rails 41, 41 may extend in parallel with each other.

So-called air bearing surfaces 43, 44, 45 are defined on the top surfaces of the front rail 37, the rear side rails 38 and the rear center rail 39, respectively. The air bearing surfaces 43, 44, 45 extend within a plane extending in parallel with the base surface 33 at a position distanced from the base surface 33. Steps 46, 47, 48 are formed at the inflow ends of the air bearing surfaces 43, 44, 45 so as to connect the inflow ends to the top surfaces of the corresponding rails 37, 38, 39, respectively. Here, the steps 46, 47, 48 may have the same height.

The aforementioned electromagnetic transducer, namely a read/write head element 49, is mounted on the slider body 31. The read/write head element 49 is embedded in the alumina film 36 of the slider body 31. A read gap and a write gap of the read/write head element 49 are exposed at the air bearing surface 45 of the rear center rail 39. A DLC (diamond-like-carbon) protecting film may be formed on the surface of the air bearing surface 45 for covering over the front end of the read/write head element 49.

Chamfers, namely curved surfaces 51, 51, are formed at the corners of the bottom surface 32 or base surface 33 near the outflow end of the slider body 31, respectively. The curved surfaces 51 have a predetermined curvature. The individual curved surface 51 is connected to the base surface 33, the side surface of the flying head slider 21 and the outflow end surface of the flying head slider 21. The curved surfaces 51 extend over the base mass 35 and the alumina film 36. Chamfering process is applied to so as to form the curved surfaces 51. The chamfering process will be described later in detail.

The airflow 34 is generated along the surface of the rotating magnetic recording disk 13. The airflow 34 flows along the bottom surface 32 of the slider body 31. The steps 46, 47, 48 serve to generate a relatively large positive pressure or lift on the air bearing surfaces 43, 44, 45, respectively. A negative pressure is generated behind the front rail 37. The negative pressure is balanced with the lift so as to keep the attitude of the flying head slider 21 in a pitch angle ox. The term “pitch angle” is used to define an inclined angle in the longitudinal direction of the slider body 31 along the direction of the airflow. The slider body 31 allows its outflow end to get closest to the magnetic recording disk 13.

The curved surfaces 51 are formed so as to round the sharp vertexes at the corners of the base surface 33 in the flying head slider 21. Even when the curved surfaces 51 of the flying head slider 21 collide against the magnetic recording disk 13, the flying head slider 21 and the magnetic recording disk 13 are sufficiently prevented from damages. The shock resistance of the hard disk drive 11 is thus sufficiently enhanced.

In the case where the sharp vertexes collide against the magnetic recording disk 13, particles of Al₂O₃—TiC possibly fall off the corners of the flying head slider 21 in response to the impact of the collision. The curved surfaces 51 in place of the sharp vertexes sufficiently serve to prevent the particles of Al₂O₃—TiC from falling off the slider body 31.

Moment is generated in the flying head slider 21 in response to the reaction of the collision of the curved surfaces 51 against the magnetic recording disk 13, for example. The moment is reduced as compared with the case where the flying head slider 21 collides against the magnetic recording disk 13 at the sharp vertexes. The flying head slider 21 is well prevented from suffering from a change in the attitude of the flying head slider 21.

Now, a brief description will be made on a method of making the flying head slider 21. A wafer bar is first cut out of a wafer. Two or more read/write head elements 49 are formed on the wafer bar. The cutting surface of the wafer bar is shaped as the bottom surfaces 32 based on photolithography, for example. The front rails 37, the rear side rails 38 and the rear center rails 39 are formed. The wafer bar is then divided into the individual slider bodies 31.

As shown in FIG. 3, a laser beam 61 is radiated to the corner of the back surface of the slider body 31. The slider body 31 defines the back surface at the back of the front surface functioning as the bottom surface 32. The laser beam forms a circular spot on the back surface of the slider body 31. The laser beam 61 is radiated to the base mass 35 at a position closest to the boundary between the base mass 35 and the alumina film 36, for example. The laser beam 61 is radiated during a predetermined duration of time. The laser beam 61 is a YAG laser beam, for example. The base mass 35, namely the Al₂O₃—TiC body, is molten on and around the beam spot of the laser beam 61.

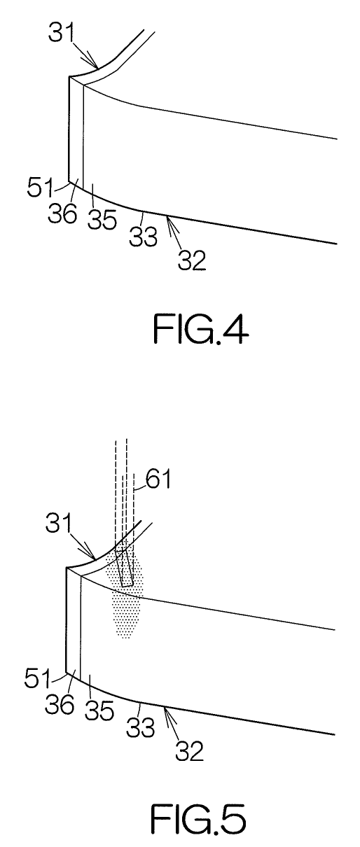

Once the radiation of the laser beam 61 is completed, the molten Al₂O₃—TiC immediately gets cured or hardened in the slider body 31. The hardening of the Al₂O₃—TiC serves to generate shrinkage stress in the slider body 31. As shown in FIG. 4, the corner of the slider body 31 thus warps back. The corner of the bottom surface 32 is thus chamfered. The curved surface 51 is formed on the corner of the bottom surface 32 of the slider body 31. The flying head slider 21 is in this manner produced.

The aforementioned method employs the radiation of the laser beam 61 for chamfering the slider body 31. The corner of the slider body 31 is molten. The curved surface 51 is formed based on the molten state of the slider body 31. The shape of the curved surface 51 can clearly be observed. The chamfering process can be repeated until a desired shape is obtained. In this manner, the slider body 31 can readily be chamfered with a high accuracy.

The radiation conditions, such as duration of irradiation, energy of irradiation, the number of irradiation, and the like, of the laser beam 61 can be specified for establishment of a desired shape of the curved surface 51. It is thus possible to form the curved surface 51 of the slider body 31 with a high accuracy in accordance with the radiation conditions. Such radiation conditions enable establishment of the curved surface 51 of a desired uniform shape on a plurality of slider bodies 31.

No dust is generated in the process of melting the slider body 31. The slider body 31 is prevented from suffering from adherence of dust to the bottom surface 32 of the slider body 31, for example. On the other hand, lapping process requires abrasive grains or particles to rub off a slider body, for example. Dust is thus generated. Adhesion of the dust to the bottom surface 32 is inevitable. The bottom surface 32 is contaminated.

As shown in FIG. 5, the laser beam 61 may form a line of exposure. The laser beam 61 may be radiated on an oblique line intersecting the edges of the base surface 33 merging at the vertex, for example. A prism lens may be utilized for the radiation, for example. The curved surface 51 is thus formed on the corner of the slider body 31 in the same manner as described above.

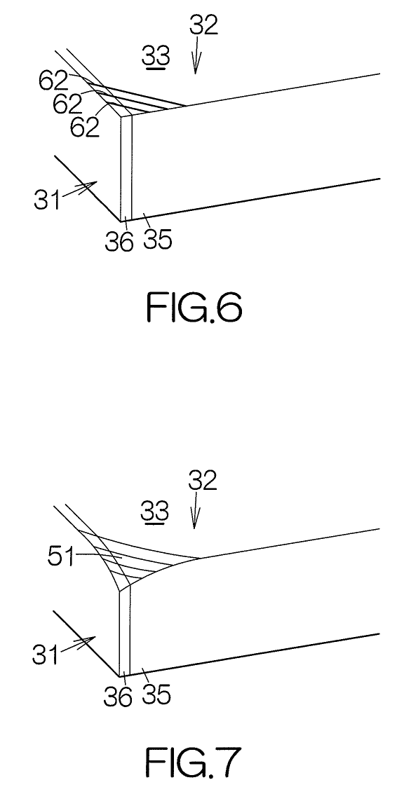

As shown in FIG. 6, slits, namely parallel grooves 62, may be formed on the slider body 31 at the corner of the bottom surface 32 for establishment of the curved surface 51. A focused ion beam may be radiated on the slider body 31 so as to form each of the grooves 62. The individual groove 62 may be formed along an oblique line intersecting the edges of the base surface 33 merging at the vertex.

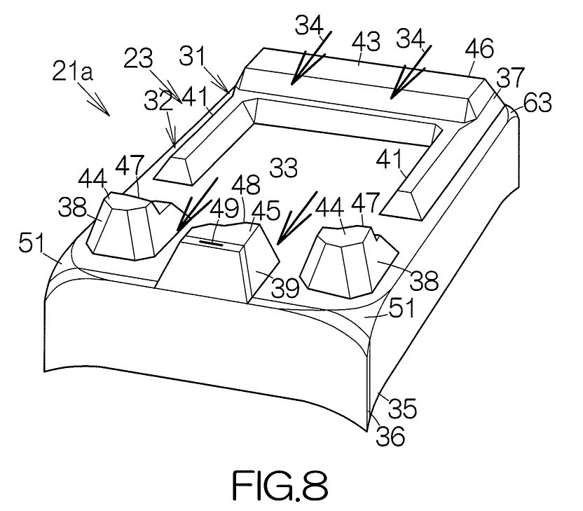

The groove or grooves 62 enable release of residual stress on the bottom surface 32 of the slider body 31 at the surfaces of the base mass 35 and the alumina film 36. The corner of the bottom surface 32 thus curves, as shown in FIG. 7. The bottom surface 32 is correspondingly chamfered. The curved surface 51 is formed in the slider body 31. The flying head slider 21 is in this manner produced.

A focused ion beam is employed to chamfer the corner of the bottom surface 32 of the slider body 31. The grooves 62 are formed at the corner of the slider body 31. The curved surface 51 is formed based on the groove 62. The shape of the curved surface 51 can clearly be observed. The chamfering process can be repeated until a desired shape is obtained. In this manner, the slider body 31 can readily be chamfered with a high accuracy.

Conditions, such as the length of the groove or grooves 62, the width of the groove or grooves 62, the position of the groove or grooves 62, and the like, can be specified for establishment of a desired shape of the curved surface 51. It is thus possible to form the curved surface 51 of the slider body 31 with a high accuracy in accordance with the conditions. Such conditions enable establishment of the curved surface 51 of a desired uniform shape on a plurality of slider bodies 31.

As shown in FIG. 8, curved surfaces 63, 63 may also be formed at the remaining corners of the slider body 31 near the inflow end of the slider body 31. In other words, all the four corners of the bottom surface 32 may be chamfered. Even when the curved surfaces 51, 63 of the flying head slider 21a collide against the magnetic recording disk 13 in the same manner as described above, the flying head slider 21a and the magnetic recording disk 13 is sufficiently prevented from suffering from damages.

What is claimed is:

1. A method of making a head slider, comprising at least radiating a laser beam to a corner of a back surface of the head slider having a front surface defining a medium-opposed surface.

2. The method according to claim 1, wherein the head slider is made of a material melting in response to exposure to the laser beam, the material generating shrinkage stress when the material gets hardened.

3. The method according to claim 1, wherein the laser beam forms a circular spot on the back surface of the head slider.

4. The method according to claim 1, wherein the laser beam forms a line of exposure on the back surface of the head slider.

5. A method of making a head slider, comprising forming a slit at a corner of a medium-opposed surface of the head slider.

6. The method according to claim 5, wherein a focused ion beam is radiated to the medium-opposed surface so as to form the slit.

7. A head slider comprising:
a slider body having a medium-opposed surface; and
a chamfer formed at a corner of the medium-opposed surface near an outflow end of the slider body based on radiation of a laser beam.

8. The head slider according to claim 7, wherein the chamfer is a curved surface.

9. A storage apparatus including at least a head slider, the head slider comprising:
a slider body having a medium-opposed surface; and
a chamfer formed at a corner of the medium-opposed surface near an outflow end of the slider body based on radiation of a laser beam.

10. The storage apparatus according to claim 9, wherein the chamfer is a curved surface.
